(19) United States
(12) Patent Application Publication
(10) Pub. No.: US 2006/0093561 A1
(43) Pub. Date: May 4, 2006

(54) Method of treating microorganisms in the oral cavity

(76) Inventor: John Kennedy, Guelph (CA)

Correspondence Address: TORYS LLP, 79 WELLINGTON ST. WEST, SUITE 3000, TORONTO, ON, M5K 1N2, CA

(21) Appl. No.: 10/978,424

(22) Filed: Nov. 2, 2004

(51) Int. Cl. A61K 8/49; A61K 31/555; A61K 31/5415; A61K 31/198

(52) U.S. Cl. 424/49; 514/185; 514/224.8; 514/561

(57) ABSTRACT

Method and kit for treating microorganisms in the oral cavity. Such microorganisms include those which contribute to periodontal disease and halitosis. The method comprises the steps of applying a photosensitizer to the interior of the mouth within and/or outside of the periodontal pockets, waiting a predetermined period of time; and irradiating the whole interior of the mouth with a non-coherent light having a wavelength spectrum absorbable by the photosensitizer, and at a predetermined light intensity and for a predetermined time period sufficient to deliver a predetermined light dose.

### FIELD OF THE INVENTION

The invention relates to methods of treating microorganisms in the oral cavity employing photodynamic therapy (PDT).

### BACKGROUND OF THE INVENTION

Oral microorganisms are the cause of many undesirable conditions including periodontitis and halitosis (i.e. bad breath). Periodontitis is associated with colonization of predominantly Gram-negative microorganisms, including the black-pigmented anaerobes Porphyromonas gingivalis and Prevotella intermedia. Halitosis is typically a consequence of volatile sulphur compounds (VSC). VSCs are produced by oral anaerobic Gram-positive bacteria by degradation of sulphur containing proteinaceous substrates in the saliva. The VSCs are released into the oral environment where they are mixed with air expired from the lungs resulting in a unpleasant oral odour. The above mentioned proteinaceous substrates may come from the intake of foods, such as meat, fish, spices, vegetables, dairy products, etc. Volatile Sulphur Compounds such as, for instance, diallyl sulfide (a thioether), can be found in garlic, which is known to cause bad breath.

Current antimicrobial treatment for periodontitis often is invasive and painful and involves a combination of scaling and root planing (SRP) and either systemic or locally delivered antibiotics, as well as surgery. Although a proven therapy, mechanical removal of calculus with its associated biofilms is laborious, does not completely eliminate offending microorganisms, and may predispose the patient to bacteremia. Furthermore, continued use of antibiotics may prompt the development of resistant bacterial strains. In an effort to bypass these problems, alternative methods of antimicrobial treatment for periodontitis have been and are being investigated.

Several groups have found that either argon or helium-neon (He—Ne) laser light irradiation can suppress periodontal pathogens, and may even decontaminate inflamed sites around implants (Wilson M, Dobson J, Sarkar S. Sensitization of periodontopathogenic bacteria to killing by light from a low-power laser. Oral Microbiology and Immunology 1993:8:182-187; Dobson J, Wilson M. Sensitization of oral bacteria in biofilms to killing by light from a low-power laser. Archives in Oral Biology 1992:37:883-887; Henry C A, Dyer B, Wagner M, Judy M, Matthews J L. Phototoxicity of argon laser irradiation on biofilms of Porphyromonas and Prevotella species. Journal of Photochemistry and Photobiology 1996:34:123-128; Moritz A, Schoop U, Goharkhay K, Schaver P, Doertbudak O, Wernisch J, Sperr W. Treatment of periodontal pockets with a diode laser. Lasers in Surgery and Medicine 1998:22:302-311; Henry C A, Judy M, Dyer B, Wagner M, Matthews J L. Sensitivity of Porphyromonas and Prevotella species in liquid media to argon laser. Photochemistry and Photobiology 1995:61:410-413.). Therefore, this approach, termed photodynamic therapy (PDT), or photoactive chemical therapy (PACT), is believed to be useful in treating periodontitis. PDT typically requires two key components, a light source and a photoreactive drug (photosensitizer) capable of binding or being in close proximity to the targeted cells. When used herein, the term photosensitizer means a molecule that absorbs light to enter an excited, highly reactive state, enabling it to catalyse the formation of reactive oxygen species (ROS) that are able to damage cell membranes and DNA.

Synthetic photosensitizers, including methylene blue, toluidine blue O (TBO), and other newly synthesized chemicals can absorb red laser light and are bactericidal for multiple species. TBO is a cell membrane active photosensitizer. To date, TBO is the drug of choice in studies focusing on PDT susceptibility of P. gingivalis, and each study has demonstrated marked reductions in viable bacteria following laser-based PDT. However, a drawback of using lasers as the light source is the inherent safety concerns associated with laser therapy and the requirement for individual pocket irradiation, which may be somewhat invasive, laborious and time-consuming. This makes it a less attractive alternative to other available antimicrobial therapies.

Successful treatment of halitosis consists of eliminating or controlling the underlying cause. Proper diet and dental hygiene are often helpful. Mouthwashes and scented toothpastes mask the condition but do not alleviate it.

While there are treatment methods for periodontitis, halitosis and other conditions caused by oral microorganisms, there is still a need for additional treatment methods which:
are non-invasive and induce less discomfort;
do not compromise the health and integrity of non-infected tissue;
reduce or eliminate the use of antibiotics which may lead to gastrointestinal problems and bacterial resistance; and
reduce labour and costs.
   The present invention is intended to meet these needs.

### SUMMARY OF THE INVENTION

In accordance with a first aspect, the invention provides a method of treating microorganisms in the oral cavity, including those causing periodontal disease, comprising the steps of:
(a) applying a photosensitizer into periodontal pockets in the mouth;
(b) applying a photosensitizer to the interior of the mouth external to the periodontal pockets and including the tongue, buccal mucosa and gum regions;
(c) waiting a predetermined period of time;
(d) irradiating the whole interior of the mouth with a non-coherent light having a wavelength spectrum absorbable by the photosensitizer, and at a predetermined light intensity and for a predetermined time period sufficient to deliver a predetermined light dose.

All or a portion of the steps may be repeated as often as required at predetermined intervals until the target microorganism populations are reduced to a desired level or eliminated. For example, the steps (i) (a), (c) and (d); or (ii) (b), (c) and (d); or all steps may be repeated every day, or every 2 days, or every 3 days.

Irradiation of the whole interior of the mouth in step (d) is performed by manipulating the light emitting treatment device such that all accessible interior surfaces are irradiated. This task may be simplified by using light sources capable of delivering light to a wide area inside the mouth and, in some cases, it will be sufficient to irradiate only such wide areas, rather than every accessible area. Optionally, individual pockets may be irradiated directly to supplement the whole mouth irradiation.

The predetermined wait period in step (c) may be from about 1 to about 60 minutes, or from about 5 to about 20 minutes, or from about 10 to about 20 minutes.

The photosensitizer may be chosen from toluidene blue, methylene blue, arianor steel blue, tryptan blue, crystal violet, azure blue cert, azure B chloride, azure 2, azure A chloride, azure B tetrafluoroborate, thionin, azure A eosinate, azure B cosinate, azure mix sicc, azure II eosinate, haematoporphyrin HCl, haematoporphyrin ester, aluminium disulphonated phthalocyanine, chlorins, photoactive fullerenes (e.g. C16-b), aminolevulinic acid (ALA), and mixtures thereof. Preferably, the photosensitizer is toluidene blue or methylene blue.

The photosensitizer may be present in a concentration of from about 2 μg/ml to about 500 μg/ml, or from about 10 μg/ml to about 50 μg/ml, or from about 10 μg/ml to about 15 μg/ml.

The photosensitizer composition for use in step (a) comprises a suitable carrier to improve adhesion to and within the periodontal pockets. The carrier is preferably a gel carrier, but may also be in the form of a cream or paste. The carrier is one which has a transmittance effective to transmit light of wavelengths absorbable by the photosensitizer and the desired rheology, pH, absorbability, and the like and may include at least one of propylene glycol, polyethylene glycol, ethanol and glycerin, and a guar hydroxypropyl derivative.

The photosensitizer composition for use in step (b) comprises a suitable carrier and is in the form of an oral rinse, atomized spray, or mouthwash which may comprise at least one of polypropylene glycol, polyethylene glycol, ethanol and glycerin in a concentration of from 2 to 20% w/w, more preferably from 5 to 10% w/w, of the solution. It may also comprise a guar hydroxypropyl derivative. It is preferred but not essential that the the composition of step (b) is less viscous than the composition of step (a).

The light used in step (d) may have a peak wavelength ranging from about 600 nm to about 730 nm, or from about 610 nm to about 690 nm, or from about 620 nm to about 640 nm, or from about 650 nm to about 680 nm.

The intensity of the light used in step (d) may range from about 10 mW/cm² to about 300 mW/cm², or from about 10 mW/cm² to about 200 mW/cm², from about 10 mW/cm² to about 150 mW/cm², from about 25 mW/cm² to about 100 mW/cm², or from about 95 mW/cm² to about 105 mW/cm².

The light dose used in step (d) may range from about 2 J/cm² to about 60 J/cm², or from about 2 J/cm² to about 30 J/cm², or from about 6 j/cm² to about 25 j/cm², or from about 6 J/cm² to about 12 J/cm².

The microbial organisms that may be targeted include but are not limited to Porphyromonas gingivalis 381; Prevotella intermedia 25611; Actinobacillus actinomycetemcomitans UT32; Fusobacterium nucleatum 1213; and Bacteroides forsythus 43037.

The invention further provides, in accordance with a second aspect, a method of treating halitosis comprising the following steps which may be repeated at predetermined intervals as often as is required until the symptoms of halitosis are reduced to a desired level or eliminated:
(a) applying a photosensitizer to the interior of the mouth, including the tongue, buccal mucosa and gum regions, and, optionally, the interior of the periodontal pockets;
(b) waiting a predetermined period of time; and
(c) irradiating the whole interior of the mouth with a non-coherent light having a wavelength spectrum absorbable by the photosensitizer, and at a predetermined light intensity and for a predetermined time period sufficient to deliver a predetermined light dose.

In accordance with a third aspect, the invention provides a kit for treating microorganisms in the oral cavity comprising:
(a) an effective concentration of a photosensitizer;
(b) at least one light emitting treatment device operable to emit non-coherent light at a wavelength spectrum absorbable by the photosensitizer and at a predetermined light intensity;
(c) instructions for performing the method according to the first and/or second aspects of the invention, including instructions concerning the time of irradiation in each irradiation step whereby the predetermined light dose is delivered in each of these steps.

In accordance with a fourth aspect, the invention provides the use of a light emitting treatment device operable to emit non-coherent light in combination with a photosensitizer to inactivate oral microorganisms, including those that cause various forms of periodontitis as well as halitosis, the light having a wavelength spectrum absorbable by the photosensitizer and a peak wavelength that may range from about 600 nm to about 730 nm, or from about 610 nm to about 690 nm.

Still other objects and advantages of the present invention and methods of construction of same will become readily apparent to those skilled in the art from the following detailed description, wherein only the preferred embodiments are described, simply by way of illustration of the best mode contemplated of carrying out the invention. As will be realized, the invention is capable of other and different embodiments and methods of construction, and its several details are capable of modification in various obvious respects, all without departing from the invention. Accordingly, the drawings and description are to be regarded as illustrative in nature, and not as restrictive.

### BRIEF DESCRIPTIONS OF THE DRAWINGS

FIG. 1 illustrates the level of kill of P. gingivalis under various in vitro conditions. P. gingivalis was incubated in 50 μg/ml TBO for 5 minutes prior to irradiation by either the red-filtered xenon lamp or He—Ne laser (2.2 J/cm²). The symbol “*” indicates a statistically significant (p<0.05) decline in bacterial survival compared to respective No TBO controls. The symbol “o” indicates a statistically significant decline in bacterial survival compared to He—Ne Laser treated sample (p=0.028). Mean±SD; n=3.

FIG. 2 illustrates the effect of varying xenon-lamp light intensities on P. gingivalis survival. P. gingivalis was incubated with 50 μg/ml TBO for 5 minutes prior to xenon lamp irradiation at either 10, 25 or 100 mW/cm² respectively (total light dose 2.2 J/cm²) with No TBO controls used for comparison. Each light intensity produced a significant decline in P. gingivalis survivors (p<0.05) compared to their respective controls. The symbol “*” indicates a significant increase in bacterial killing compared with the 10 mW/cm² light intensity treatment with TBO (p<0.05). Mean±SD; n=3.

FIG. 3 illustrates the effect of altered xenon-lamp light doses on P. gingivalis survival in the presence and absence of TBO. Each sample of P. gingivalis was preincubated with TBO (50 μg/ml) and irradiated with 0, 2.2, 5, 6.3, 8.3 or 10 J/cm². Each light dose induced a statistically significant decline in bacterial survivors (p<0.05). TBO absent controls did not produce a significant relationship between light dose and bacterial killing (r²=0.256). With the inclusion of TBO a linear relationship developed such that an increasing light dose led to increased bacterial killing (r²=0.916) from 2.2 to 10 J/cm². At 10 J/cm² the detectable limit of the assay was reached (2.48 log cfu/ml). The linear relation could not be extended from 0 to 2.2 J/cm². Mean±SD; n=3.

FIG. 4 illustrates the effect of various conditions on the survival of serum suspended P. gingivalis. The FBS/P. gingivalis suspension was treated at 6.3 J/cm² and 100 mW/cm² using a non-laser xenon lamp following incubation with 12.5 μg/ml TBO. The symbol “*” indicates a significant decline in bacterial survival from controls (p<0.05). The symbol “o” indicates a significant increase in bacterial survival when compared to treatment in the absence of serum (p=0.002). Mean±SD; n=3.

FIG. 5A illustrates the effects of light dose effected by a xenon lamp on survival of blood suspended P. gingivalis. Defibrinated sheep's blood was used to resuspend P. gingivalis prior to treatment (lamp at 100 mW/cm² with 12.5 μg/ml TBO) with 0, 6.3, 10, 15 or 20 J/cm². All light doses, except the no light control, produced significant reductions in bacterial survivors (p<0.05) when TBO was present. There is a strong linear relationship from 0 to 10 J/cm² (r²=0.999) that stabilized to an approximate 3 log kill compared to TBO absent controls at higher light doses. Mean±SD; n=3.

FIG. 5B illustrates the effect of blood dilution or concentration on P. gingivalis survival following TBO incubation and radiation with a xenon lamp. Defibrinated sheep's blood was diluted to ½, ¼ and ⅛ with PBS, and a PBS only solution was used as a positive control. Following P. gingivalis resuspension with the diluted blood, the bacterial suspension was incubated for 5 min. with 12.5 μg/ml TBO and then treated with a non-laser xenon lamp light dose of 10 J/cm² and 100 mW/cm². A strong linear correlation between blood concentration and bacterial killing was realized (r²=0.967), whereby the more dilute the blood used, the higher the kill. Mean±SD; n=3.

FIG. 6 illustrates the effect of serum washout on P. gingivalis survival following PDT with a xenon lamp at varying light doses. After a one-hour incubation in FBS P. gingivalis was washed and resuspended in PBS. P. gingivalis was then treated with 12.5 μg/ml TBO and irradiated with a 6.3 or 10 J/cm² light dose at 100 mW/cm². The symbol “*” indicates a significant decline in P. gingivalis survivors compared to their respective No TBO controls p<0.05). Mean±SD; n=3.

FIG. 7 illustrates the relationship of P. gingivalis kill to TBO concentration.

FIG. 8 illustrates the effectiveness of xenon lamp initiated PDT on different oral pathogens. A. actinomycetemcomitans (Aa); B. forsythus (Bf); F. nucleatum (Fn); P. intermedia (Pi) and P. gingivalis (Pg) were incubated in FBS for one hour at room temperature. Each bacterial species was then washed and resuspended in PBS prior to treatment with 12.5 μg/ml TBO and a 100 mW/cm² 10 J/cm² light dose. P. gingivalis LD- and LD treatments were used as a comparison for killing efficacy. A. actinomycetemcomitans, F. nucleatum and P. intermedia each experienced a significant decline in survivors (p<0.05) that was comparable to P. gingivalis killing. The symbol “*” indicates that only B. forsythus exhibited a significantly higher survival rate than P. gingivalis (p=0.039). Mean±SD; n=3.

FIG. 9 illustrates the attenuation of light traveling through gingival tissue as measured in a patient. Transillumination spectroscopy was performed by delivering white light to the gingival exterior and collecting light intensity measurements via a fiber optic probe placed inside the gingival pocket. Six locations in the mouth of this subject were examined. The results indicated that for the spectral region of 600 to 1000 mm, approximately 10% to 50% of the light incident on the outer gingival tissue penetrated into a typical 5-7 mm pocket.

The bottom image illustrates the relative placement of the light source to the fiber optic probe placed inside the gingival pocket. The light source was a fiber coupled tungsten halogen lamp coupled to an “elbowed” light guide which emitted ˜5 mW of power over a spot with a 3 mm diameter. To determine the absolute attenuation by the tissue, the light source and the fiber optic probe were calibrated against known intensities of light. Liquid tissue was comprised of a scattering material (Intralipid) and a dye (India ink) in order to match typical tissue optical properties. The liquid tissue simulated phantoms of known absorption and scattering properties. Measurements of the intensity of light were made at several distances from the source fiber. Monte Carlo simulations were conducted using the light intensity values measured throughout the phantom. To determine the correction factor needed between the measured signal and the actual intensity of light, the results of the Monte Carlo simulations were compared to the signals obtained by the detector.

### DETAILED DESCRIPTION OF THE INVENTION

In accordance with a first embodiment of the invention for the treatment of periodontal disease, a photosensitizer composition is applied in gel form to periodontal pockets in the mouth. The composition comprises (i) at least one photosensitizer (which may include a mixture of photosensitizers), such photosensitizers including at least one of toluidine blue (TBO; also known as tolonium chloride), methylene blue, or any other photosensitizer determined to be effective, (ii) a gel carrier comprising propylene glycol and, optionally, (iii) other orally suitable ingredients. The gel carrier allows simple and stable delivery of the photosensitizer into periodontal pockets as well as to mucosal surfaces so that it would not be washed out prior to light activation and has a transmittance effective to transmit light of wavelengths absorbable by the photosensitizer. The photosensitizer is present in a concentration of 12.5 μg/ml. The photosensitizing composition is introduced using a syringe; however, any other suitable device such as a cannula can be used.

Either before or after this step, a less viscous photosensitizing composition in the form of a mouthwash or oral rinse solution is introduced to all accessible interior surfaces of the mouth external to the periodontal pockets, including the tongue, buccal mucosa and gum regions. The mouthwash also includes 12.5 μg/ml of the photosensitizer in an orally acceptable solution. The patient gargles the mouthwash and holds it in his or her mouth for a tolerable period of time (i.e. from about 30 seconds to a minute). This process is repeated from 1 to 5 times to maximize exposure of oral tissues to the photosensitizer. It will be appreciated that the solution may be applied in other suitable ways such as by manual or assisted irrigation.

After waiting a predetermined period of time to permit the photosensitizer to adhere to or be absorbed by the oral microorganisms, which in this embodiment is 3 to 20 minutes, a light emitting treatment device is used to irradiate the whole mouth (including the periodontal pockets) to activate the photosensitizer applied inside and outside of the periodontal pockets. The light device emits non-coherent light having a wavelength spectrum matching the absorption curve of the photosensitizer. TBO and methylene blue can absorb light in the red region having a peak wavelength ranging from about 610 nm to about 690 nm. In this embodiment, the wavelength of light emitted by the light emitting diodes peaks at 633 nm when using TBO or about 668 nm when using methylene blue. Such light can penetrate gum tissues to activate the photosensitizer contained in the periodontal pockets. The device is adapted to deliver light to all regions of the oral cavity, including under the tongue and through the flesh covered lingual, labial, anterior and posterior areas of the oral cavity and through the bite surface.

The light intensity of the light device is 100 mW/cm² and the irradiation time is about 100 seconds for each surface such that a light dose of at least 10 J/cm² is delivered. The device is connected to a microprocessor which is used to control the light intensity and time of illumination, and therefore also the light dose. By using a computer-based approach to light delivery, the consistency and repeatability of the method can be enhanced. In this embodiment, the device is sterilized after each use using a suitable disinfectant. However, alternative light devices could be either disposable or bagged for infection control purposes.

After the initial illumination step, the patient gargles once again with the photosensitizing mouthwash solution and the entire mouth is irradiated again, using the same light device to deliver the same light dose, to treat resident microbial organisms or those which might have been released following mechanical procedures, such as scaling or root planing. Such microbial organisms may cause reinfection of the treated periodontal pockets and typically include the following organisms: Porphyromonus gingivalis 381; Prevotella intermedia 25611; Actinobacillus actinomycetemcomitans UT32; Fusobacterium nucleatum 1213; and Bacteroides forsythus 43037.

The entire procedure or portions thereof may be repeated at predetermined intervals until symptoms of bacterial infection are reduced to a desired level or eliminated. For example, the patient can gargle with a photosensitizer mouthwash solution at home and self-irradiate using a home kit according to the present invention (described further below) every 3 days until the symptoms of periodontal disease are reduced to the desired level. Optionally and/or additionally, the patient can return to the dentist's office to have the periodontal pockets filled with the photosensitizer gel composition followed by further transdermal and transtooth irradiation.

In accordance with another preferred embodiment of the invention, there is provided a method of killing bacteria responsible for halitosis. Such method involves gargling with the above described photosensitizer based mouthwash solution, waiting 5 to 20 minutes and then and irradiating the whole interior of the mouth using the light emitting device. Again, light is applied for at least 1 to 10 minutes to all accessible surfaces such that a light dose of at least 10 J/cm² is delivered. These steps are repeated every day until the symptoms are reduced to a desired level. Alternatively or additionally, these steps may be performed every day to keep halitosis causing bacteria to acceptable levels.

A third preferred embodiment of the invention is a kit for use in treating microorganisms in the oral cavity. The kit can be designed for professional or home use. In the present embodiment designed for home use, the kit comprises the photosensitizer mouthwash solution described above, the above described light emitting device, and instructions for using the components to reduce oral microorganisms to acceptable or desired levels. The instructions, in this case, would direct the user to perform the above described method of gargling with the mouthwash solution, waiting the predetermined period of time, and irradiating all accessible surfaces within the oral cavity for the minimum period required to deliver the minimum light dose.

A kit designed for professional use may further include the photosensitizer in gel form (as described above), a syringe, cannula or other suitable device for applying the photosensitizer gel to the periodontal pockets, and additional instructions for performing the method according to the first preferred embodiment of the invention described above. The kit may also include two or more alternative light sources designed to reach different areas within the oral cavity.

Alternative light sources include light emitting treatment devices capable of irradiating large portions of the oral cavity at once, such as those described in U.S. Pat. No. 5,487,662 to Kipke at al., U.S. Pat. No. 4,867,682 to Hammesfahr et al., U.S. Pat. No. 5,316,473 to Hare and U.S. Pat. No. 4,553,936 to Wang. Other light emitting treatment devices which can be manually manipulated to deliver light to various regions in the mouth which can be used include fibre optic wands, guns or light guides, remote light engines utilizing light generation means in the form of quartz halogen, mercury xenon, xenon, metal halide, sulfur based or other light emitting diode (LED) technology, flexible lightpipes composed of a number of individual fiber optic elements or liquid lightpipes, and other dental impression trays containing light emitting diodes. While various light devices may be used, it will be appreciated that the light device must be capable of delivering an effective dose of light at an effective wavelength. Thus, higher intensities may be used in combination with pulsed light delivery, or lower intensities with continuous light delivery. The spectrum of light emitted by the light emitting treatment device would be selected to match the particular absorption curve of the photosensitizer used. A bandpass filter could be used to eliminate wavelengths not absorbed by the photosensitizer.

Preferred light emitting treatment devices are expected to be LED based as such can be made into a variety of shapes that will be comfortable for patients and simple to apply for dentists and/or dental hygienists. LED light sources will also reduce the potential for the generation of potentially uncomfortable heat, and would therefore be much more acceptable to patients. It is expected that a suitable light device can be made from a standard dental mouth plate carrying an encapsulated scattering gel (as is known in the art), which gel is pressed up against the gums when the device is in use. LEDs can be embedded directly into the gel and positioned to face the gingival tissue. The scattering medium should ensure that the light is delivered in a uniform cross-section to the gingival tissue surface. Electronic connections to the LEDs can be made to the dental plate out the front of the mouth. Alternatively, it is contemplated that the light source may be in the form of optical fibers or other light guides coupled to LEDs with their terminus within the scattering gel.

### EXAMPLES

Tests were conducted to investigate the effectiveness of a red-filtered xenon lamp, in combination with TBO, in suppressing P. gingivalis. The results were compared to results of tests employing a He—Ne laser in vitro. Further dosimetric and environmental analyses were performed using the xenon lamp to identify the optimal parameters for in vivo applications. Finally, the parameters were tested on four other species of periodontal pathogens.

Materials and Methods

Strains and Growth Conditions

The following bacteria were used in these experiments and cultivated in an anaerobic chamber (available commercially from Coy Manufacturing Co.; AnnArbor, Mich., USA): Porphyromonas gingivalis 381; Prevotella intermedia 25611; Actinobacillus actinomycetemcomitans UT32; Fusobacterium nucleatum 1213; Bacteroides forsythus 43037. P. gingivalis and P. intermedia strains were subcultured weekly in Todd Hewitt Broth (available commercially from Fisher Scientific; Nepean, Ontario, Canada; hereinafter referred to as Fisher) supplemented with 5 μg/ml hemin (Sigma-Aldrich Co.; Oakville, Ontario, Canada; hereinafter referred to as Sigma) and 1 μg/ml menadione (Sigma). A. actinomycetemcomitans was subcultured in Tryptic Soy Broth (Fisher) containing 6 g/l yeast extract (Fisher) and 8 ml of 1.5% sodium bicarbonate, filter sterilized (Fisher). F. nucleatum was subcultured in Todd Hewitt Broth. B. forsythus was subcultured in Brain Heart Infusion Broth (95 ml) (Fisher) supplemented with 0.001 g N-acetylmuramic acid (Sigma) in 5 ml Horse Serum (Cedarlane Laboratories; Hornby, Ontario, Canada), filter sterilized. Prior to experiments, a fresh broth tube was inoculated and allowed to grow overnight at 37° C. in the anaerobic chamber.

Light Sources

A 100 Watt red-filtered xenon fibre optic light source having a bandwidth of 620-640 nm (available commercially from EXFO, Inc.; Mississauga, Ontario, Canada) was used in these experiments. Laser light irradiation was provided by a diode laser at 635 nm (sold by High Powered Devices). Light dose for each source was calculated by the cross product of the TBO absorption spectrum and light spectrum. Hence, light doses calculated for each source were such that the photons absorbed by each source would be equivalent for a given light dose for the He—Ne laser.

Photosensitizer

The photosensitizer TBO (Sigma) was dissolved in Dulbecco's Phosphate Buffered Saline (PBS) (Sigma) to a concentration of 1 mg/ml under ambient light. The solution was filter sterilized, aliquoted and stored in dark tubes at −20° C. for up to 3 months without a loss in potency. The stock was diluted to appropriate concentrations with PBS as required.

Photodynamic Therapy Assay

An overnight bacterial culture was removed from the anaerobic chamber and centrifuged at 2400×g for 5 minutes in an IEC21000R centrifuge (Fisher). The supernatant was discarded and the pellet washed and resuspended three times in PBS. The bacterial suspension was then adjusted to a working concentration of approximately 10¹⁰ colony forming units (cfu)/ml by measurement of optical density at 600 nm (Pharmacia LKB Ultraspec III). The suspension was returned to the anaerobic chamber and aliquoted in pre-reduced 1.5 ml microfuge tubes (Fisher) in order to decrease oxygen exposure of the anaerobic suspension.

Prior to light irradiation, 75 μl of calibrated bacterial suspension (7.5×10⁸ cfu) was mixed with 75 μl of TBO stock solution (100 μg/ml) in a sterile tube, for a final mixture concentration of 5×10⁹ cfu/ml and 50 μg/ml TBO. The mixture was then incubated at room temperature for 5 min. in the dark. TBO negative controls were incubated with an equal volume of sterile PBS instead of TBO. The mixture was then added to a sterilized perfusion chamber (Cedarlane Laboratories) mounted on a glass coverslip. The perfusion chamber was 500 μm thick, and so attenuation of the treatment light by the TBO was only 30% at 50 μg/ml. For most treatments at a concentration of 12.5 μg/ml TBO, the attenuation of treatment light was only 9%. The inoculated perfusion chamber was irradiated under the desired conditions with a light beam whose diameter covered the perfusion chamber's transparent surface. Upon completion, the mixture was removed and serially diluted in PBS and each dilution was plated on the following media: for P. gingivalis, P. intermedia, and F. nucleatum, Trypticase Soy Agar (Fisher) supplemented with 5 g/l yeast extract, 5 μg/ml hemin, 1 μg/ml menadione, 50 ml/l defibrinated sheep's blood (Oxoid, Inc.; Nepean, Ontario, Canada); for B. forsythus, Trypticase Soy Agar supplemented with 4 g/L yeast extract, 4 g/L trypticase peptone (Fisher), N-acetymuramic acid (10 mg/ml stock filter sterilized into medium to 10 mg/l); and for A. actinomycetemcomitans, Tryptic Soy Agar. Once plated, P. gingivalis, P. intermedia, F. nucleatum and B. forsythus were grown in dark anaerobic jars containing 10% H₂:CO₂ and the balance N₂ (BOC Gases Canada, Ltd.; Toronto, Ontario, Canada) for 6 days at 37° C. in the presence of catalyst. A. actinomycetemcomitans was grown overnight at 37° C. in 10% CO₂ with the balance air. Colony counts determined the number of bacterial survivors and they were expressed as cfu/ml.

Serum and Blood Bacteria Suspensions

To estimate the potential of the xenon lamp to induce bacterial suppression in vivo an attempt was made to mimic some periodontal pocket conditions in vitro. The bacterial pellet was resuspended in either fetal bovine serum (FBS) or defibrinated sheep's blood after the final PBS wash and was then used in the PDT assay at a final concentration of 50% (v/v).

Serum Washout of Bacteria

PDT of a patient's periodontal pocket is likely to cause some degree of washout when TBO is added. To mimic the washing effect, the bacteria was prepared as described and the suspension was incubated for 1 hour at room temperature in an appropriately adjusted volume of sterile serum. The serum/bacteria mixture was then centrifugated and washed as before using PBS to resuspend the pellet.

Results

Comparison of Xenon Lamp and He—Ne Laser Treatment in Suppressing P. gingivalis

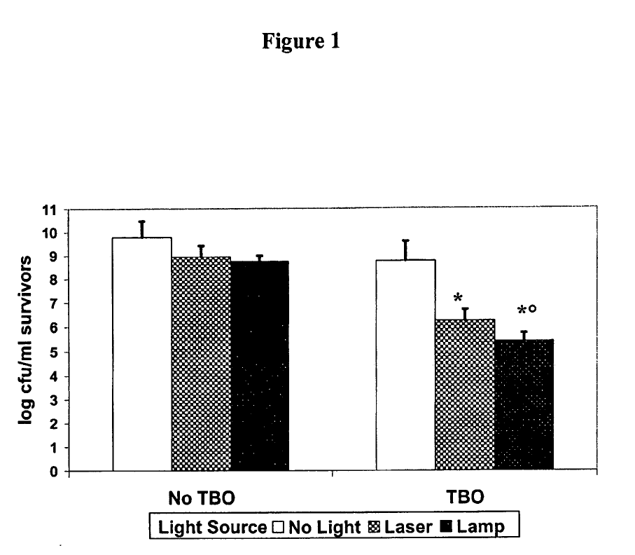

Using a light dose of 2.2 J/cm² and a TBO concentration of 50 μg/ml per mixture a killing of 2.43±0.39 logs (an approximately 270× reduction in viable bacteria) was obtained when treating with the He—Ne laser, and 3.34±0.24 logs killing was obtained with the xenon lamp (FIG. 1). This represents a near 10-fold increase in bacterial killing (Student's T-test p=0.028) and suggests that the xenon lamp is at least as efficient an inducer of the photosensitizer TBO as the He—Ne laser.

Alterations in Treatment Conditions for the Xenon Lamp

In order to better utilize the xenon lamp for PDT, experiments were performed to determine the optimal conditions in vitro for which 5 logs of bacterial killing could be achieved (a 100,000× reduction in viable bacteria). The parameters tested were light intensity (mW/cm²), light dose (J/cm²) and TBO concentration (μg/ml) using P. gingivalis as the standard test bacterium.

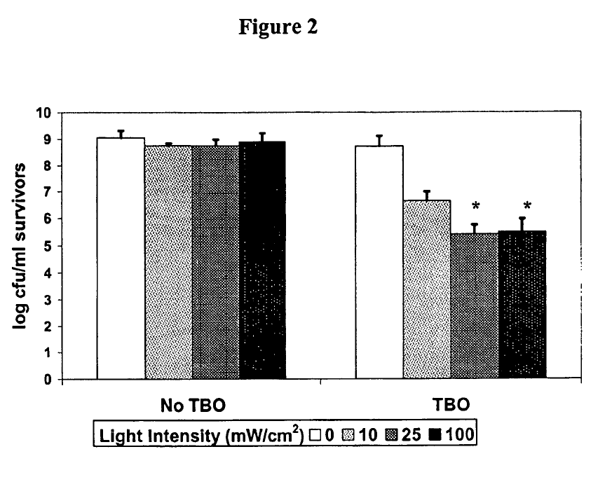

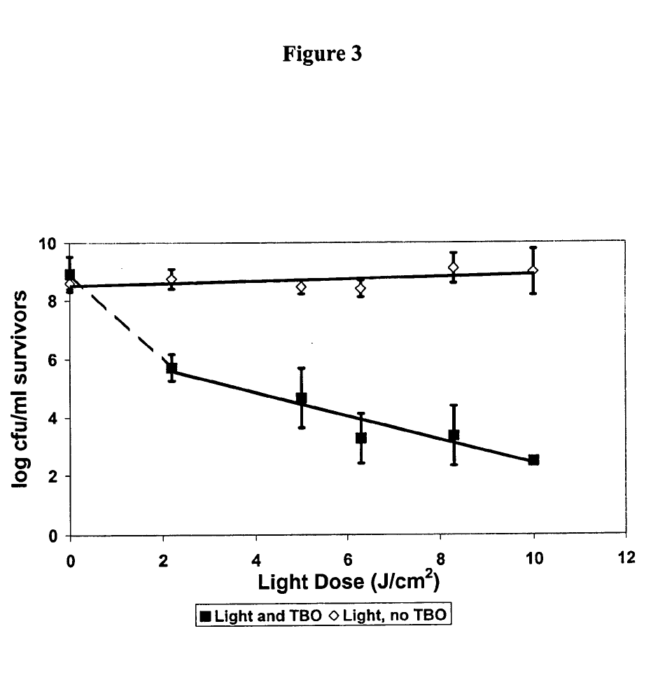

FIG. 2 illustrates the effect of altered light intensity on P. gingivalis survival. 10 mW/cm² achieved a kill of 2.12±0.36 logs; 25 mW/cm² and 100 mW/cm² each achieved a kill of over 3 logs (3.34±0.41 logs and 3.41±0.58 logs respectively). Both the 25 and 100 mW/cm² lamp intensities provided significantly higher killing compared with 10 mW/cm² (Student's T-test p=0.012 and p=0.029 respectively). There was no significant difference between 25 mW/cm² and 100 mW/cm². Under these conditions, it would appear that the effect of increased light intensity plateaus at ˜25 mW/cm². For the remainder of the study, a light intensity of 100 mW/cm² was used, as it provided the shortest irradiation time to reach desired light doses. The relationship between increased xenon lamp light dose and P. gingivalis killing is shown in FIG. 3. There was a statistically significant negative linear relationship from 2.2 J/cm² to 10 J/cm² (r=0.96); as light dose increased there was a decrease in viable bacteria. The detectable limit of the assay (300 cfu/ml or 2.48 log cfu/ml bacterial survivors) was reached at 10 J/cm², at which point no bacterial colonies were visible. The target of 5 logs bacterial kill was achieved at the lower light dose of 6.3 J/cm², which was used in later experiments.

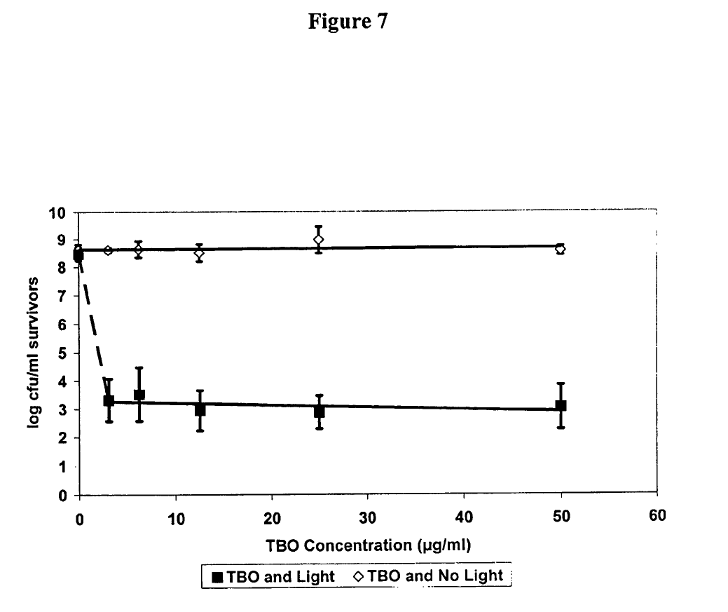

The relationship of P. gingivalis kill to TBO concentration is illustrated in FIG. 7. For each drug concentration a target of 5 logs kill was achieved at the specified light settings (6.3 J/cm² and 100 mW/cm²). This suggested that the lowest drug concentration tested was more than enough to eliminate P. gingivalis. The TBO concentration of 12.5 μg/ml was chosen for subsequent use, since killing tended to fluctuate more at the lower concentrations of 3.125 and 6.25 μg/ml.

Effect of Serum and Blood on PDT Efficacy

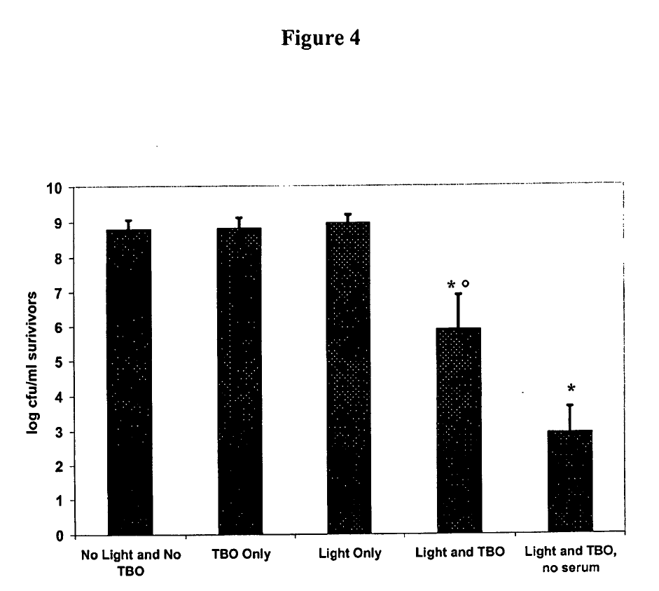

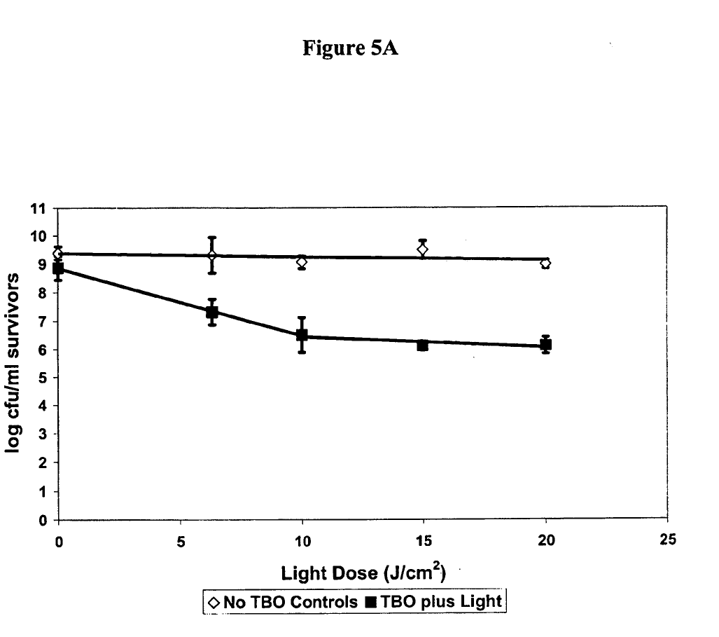

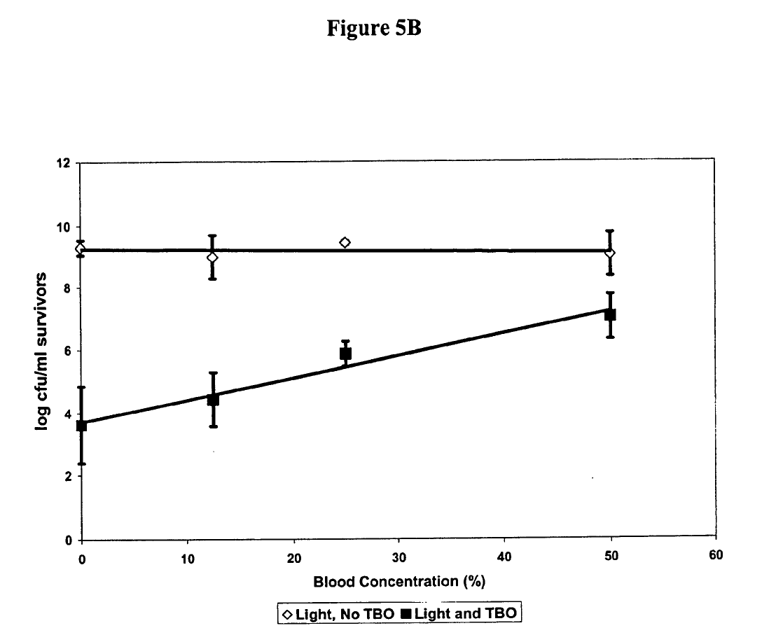

Periodontal pockets often contain serum and blood or their constituents known also as gingival crevicular fluid (GCF). To mimic these in vivo pocket conditions, bacterial pellets were resuspended in either serum or blood. Upon the addition of serum to a final concentration of 50% (v/v) prior to irradiation there was a marked increase in bacterial survivors (FIG. 4). Although a P. gingivalis kill of 6.02±0.76 logs upon treatment at the specified conditions in the absence of serum (FIG. 4) was achieved, with the addition of serum, killing was significantly attenuated to 3.05±1.02 logs. This still represented a statistically significant decline in P. gingivalis viability (Student's T-test p=0.013), in spite of exposure to a very high serum concentration. When defibrinated sheep's blood was substituted for serum, a similar result was found (FIG. 5A). Altered light doses provided a maximum blood resuspended P. gingivalis kill of 2.85±0.33 logs at a 20 J/cm². Although this was a large decrease in effectiveness compared with blood-free suspensions, there was still a statistically significant reduction in bacterial survivors (p<0.001). There was a linear relationship between light dose and bacterial killing from 0 to 10 J/cm² that appeared to level off with higher doses (FIG. 5A). Bacterial pellet resuspension in different blood dilutions resulted in progressively higher killing (FIG. 5B). Therefore blood and serum appear to partially protect P. gingivalis from PDT.

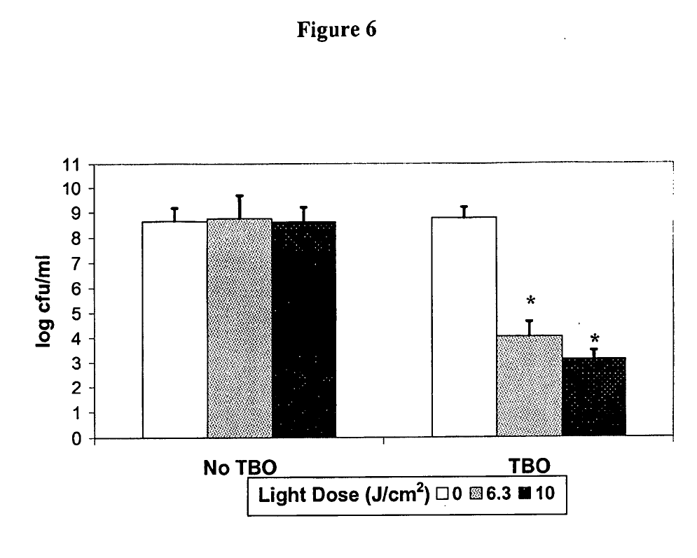

When this treatment is translated to in vivo trials, the patient's periodontal pocket is likely to experience some degree of washout when the drug is added as a rinse or pocket lavage. A bacterial suspension that had first been exposed to serum and then washed was used in the PDT assay; the results are illustrated in FIG. 6. Washing out the serum from the bacterial suspension returned the P. gingivalis kill to the 5 logs target (4.71±1.07 logs of killing at 6.3 J/cm² and 5.48±0.68 logs kill at 10 J/cm²). For further studies, a light dose of 10 J/cm² was chosen as it provided a consistent 5 logs kill.

PDT Efficacy on Various Periodontal Bacterial Species

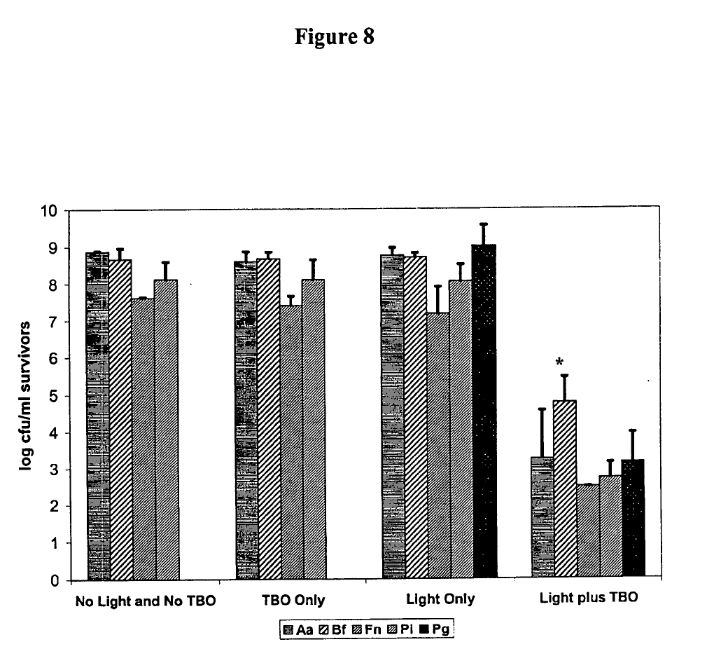

Using the serum washout protocol described, the set PDT parameters (10 J/cm², 100 mW/cm² and 12.5 μg/ml TBO) were used in tests concerning other bacterial species. An approximately 5 logs of kill for each of the following species was achieved as shown in FIG. 8: P. intermedia (5.33±0.62 logs kill); F. nucleatum (4.7±0.72 logs kill); A. actinomycetemcomitans (5.51±1.34 logs kill). Interestingly, there was a statistically significant decrease in killing of B. forsythus compared to that found for the P. gingivalis control (3.92±0.68 logs killing; p=0.0391), but it was still a significant reduction in viable B. forsythus. Overall, it would appear that PDT can be used effectively to kill periodontal pathogens when utilizing a xenon lamp at 10 J/cm², 100 mW/cm², and 12.5 μg/ml TBO, even in the presence of serum and blood at high concentrations.

Transmission of Light through Gingival Tissue

The attenuation of light traveling through gingival tissue was measured using trans-illumination spectroscopy. Measurements were made on typical pockets of patients suffering from periodontal disease. Low intensity white light was delivered to the exterior of gingival pocket sites using an elbowed light guide. Light in the pocket was collected by a cylindrical diffusing tipped optical fiber probe, inserted into the gingival pocket at depths between 4-7 mm. This light was delivered to a portable spectrometer system. The apparatus was calibrated to measurements in tissue simulating liquid phantoms of known optical properties and comparing these results with calculations of the expected light intensity in the phantoms.

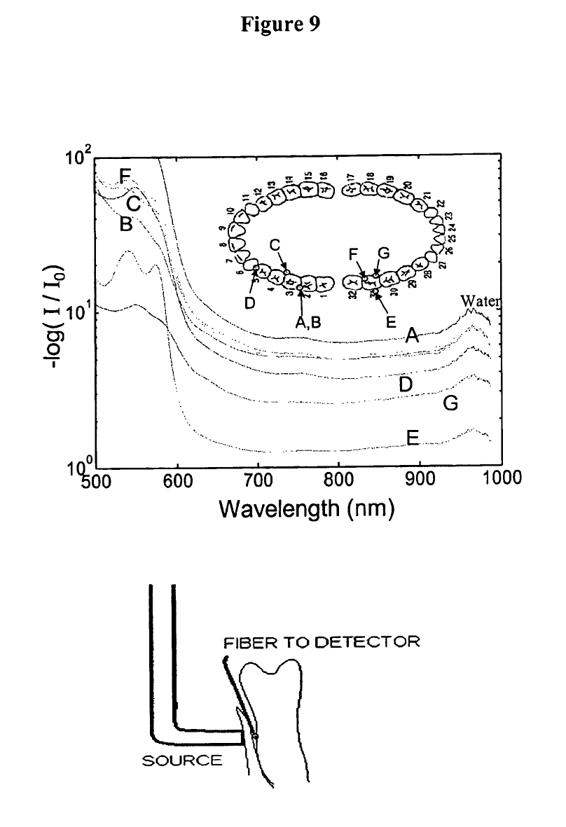

Two subjects were examined, a 35-40 year old woman and a 70-75 year old man. Both had gingival pockets that were 5-7 mm deep. Both subjects experienced minor bleeding of the pockets during the procedure. FIG. 9 shows the transmission spectra measured on one patient along with the measurement location in the mouth for each spectrum. Approximately 10-50% of the incident light penetrates into the gingival pocket, with attenuation greater for pockets located between teeth, and less attenuation for pockets located on facial or lingual sides.

Discussion

These experiments demonstrated that an alternative light source, i.e. a conventional red-filtered xenon lamp, is at least as effective an inducer of PDT as a laser and may even provide a significantly improved kill when testing P. gingivalis in vitro. The ten-fold increase in killing (shown in FIG. 1) could be due to a broad-spectrum effect provided by the xenon lamp compared to the He—Ne laser. The lamp, although filtered for light in the red spectrum, is not as precise as laser light and it was necessary to increase the lamp delivered light by 10% in order to deliver an equal amount of photons in the upper red spectrum compared to the laser. This 10% increase in light delivered would include an increase in low wavelength lamp light, which may weakly activate additional TBO molecules since the excitation wavelength of TBO excitation can vary from 620-660 nm, depending on TBO molecular stability. This additional excitation may then cause the additional bacterial killing witnessed when compared to the laser.

Light intensity and light dose were both shown to contribute significantly to bacterial killing. Light intensity would be expected to significantly affect light penetration into the periodontal pocket. For these experiments, the near maximum intensity available (100 mW/cm²) was used since higher intensities provided improved penetrance and shorter irradiation periods. A consideration in using this light intensity with the xenon lamp is the generation of heat. For in vivo use, the intensity would be selected to keep the heat generation to levels tolerable by the patient. The time of irradiation would be increased as required to deliver the required light dose.

Total light received by the TBO/bacteria mixture is a significant determinant of the amount of kill, as is evident by their linear relationship. This relationship was demonstrated previously with a He—Ne laser and reiterated in these experiments for the xenon lamp. However, from 0 to 2.2 J/cm² the relationship is skewed. It may be argued that the relationship is not linear for this light dose range. The effectiveness of light doses below 2.2 J/cm² could be limited by a threshold effect, whereby the number of photons absorbed by lower light doses is insufficient to provide killing, and above this threshold light dose cell death increases linearly. Preliminary studies have also shown that enough light can be delivered into periodontal pockets using transperiodontal illumination/irradiation that would be capable of activating TBO.

In these experiments, low concentrations of TBO provided the targeted 5 logs of kill. Such low concentrations are believed to circumvent potentially toxic effects of on human tissue.

When attempting PDT for periodontal pockets, treatment may be confounded by the presence of serum-derived periodontal crevicular fluid (GCF) as well as blood. PDT, under the conditions described, provided a significant bacterial kill of 5 logs in the absence of serum, but decreased sharply in the presence of 50% FBS. Similarly, decreased bactericidal activity was evident when defibrinated sheep's blood was used. Yet, increased light dose increased killing to 3 logs in samples containing blood. The similarity between serum and blood kills would suggest that it is the serum itself that provides protection to P. gingivalis from TBO, since serum is a major blood component. The protection could be due to the presence of light scattering/absorbing proteins—simple opacity may limit light penetration into the sample. Indeed, dilution of blood resulted in significantly greater killing (FIG. 5B), suggesting the protective effect does depend on light attenuation, since light can more readily penetrate diluted samples (data not shown). An alternative explanation may be that a serum component(s) binds to P. gingivalis and protects it from activated TBO. However, while this may apply to B. forsythus (see below) the washout experiments suggest that the latter is not the case. Following exposure to serum, washed bacterial suspensions all exhibited significant killing that was no different from serum-free conditions. Hence, the opacity of contaminating serum or blood must be of concern in PDT targeted at periodontal pockets in vivo. However, when delivering dilute TBO solutions in vivo one would expect a diluting effect that would diminish GCF to acceptable concentrations, well below those tested here with serum and blood.

All four bacterial species exposed to PDT exhibited significant killing with only B. forsythus deviating from the targeted 5 logs kill. Reasons for the relatively reduced sensitivity of B. forsythus were not determined. It might bind serum proteins more efficiently, or it may be able to counter the harmful effects of oxygen radicals generated. Other Bacteroides spp. have the ability to maintain critical cellular enzymes in a stable inactive state in the presence of oxygen and the ability to repair damaged enzymes and DNA once the organism is returned to anaerobic conditions. These traits may also aid B. forsythus survival against PDT, although this has not yet been demonstrated. The slightly higher survival rate of B. forsythus may not necessarily detract from the use of PDT. It has been suggested that B. forsythus has an ecologically dependent relationship with P. gingivalis and requires other bacteria such as F. nucleatum in its food web. Therefore, significant suppression of other periodontal bacterial species should hamper B. forsythus survival as well.

The above described embodiments of the invention are merely descriptive of its principles and are not to be considered limiting. Further modifications of the invention herein disclosed will occur to those skilled in the respective arts and all such modifications are deemed to be within the scope of the invention as defined by the following claims.

1. A method of treating microorganisms in the oral cavity comprising the steps of:
(a) applying a photosensitizer into periodontal pockets in the mouth;
(b) applying a photosensitizer to the interior of the mouth external to the periodontal pockets and including the tongue, buccal mucosa and gum regions;
(c) waiting a predetermined period of time;
(d) irradiating the whole interior of the mouth with a non-coherent light having a wavelength spectrum absorbable by the photosensitizer, and at a predetermined light intensity and for a predetermined time period sufficient to deliver a predetermined light dose.

2. The method of claim 1 wherein the predetermined period of time in step (c) is from about 1 to about 60 minutes.

3. The method of claim 1 wherein steps (b), (c) and (d) are repeated at predetermined intervals until symptoms of bacterial infection are reduced to a desired level or eliminated.

4. The method of claim 3 wherein said predetermined interval is 1 to 3 days.

5. The method of claim 1 wherein steps (a) through (d) are repeated at predetermined intervals until symptoms of bacterial infection are reduced to a desired level or eliminated.

6. The method of claim 5 wherein said predetermined interval is 1 to 3 days.

7. The method of claim 1 wherein steps (a), (c) and (d) are repeated at predetermined intervals until symptoms of bacterial infection are reduced to a desired level or eliminated.

8. The method of claim 7 wherein said predetermined interval is 1 to 3 days.

9. The method of claim 1 wherein the photosensitizer is chosen from toluidene blue, methylene blue, arianor steel blue, tryptan blue, crystal violet, azure blue cert, azure B chloride, azure 2, azure A chloride, azure B tetrafluoroborate, thionin, azure A eosinate, azure B eosinate, azure mix sicc, azure II eosinate, haematoporphyrin HCl, haematoporphyrin ester, aluminium disulphonated phthalocyanine, chlorins, photoactive fullerenes (e.g. C16-b), aminolevulinic acid (ALA), and mixtures thereof.

10. The method of claim 9 wherein the photosensitizer is chosen from toluidene blue, methylene blue, and mixtures thereof.

11. The method of claim 9 wherein the photosensitizer is toluidene blue.

12. The method of claim 9 wherein the photosensitizer is methylene blue.

13. The method of claim 1 wherein the photosensitizer forms part of a composition and is present in a concentration of from about 2 μg/ml to about 500 μg/ml.

14. The method of claim 13 wherein the photosensitizer is present in a concentration of from about 10 μg/ml to about 50 μg/ml.

15. The method of claim 14 wherein the photosensitizer is present in a concentration of from about 10 μg/ml to about 15 μg/ml.

16. The method of claim 1 wherein the photosensitizer is part of a composition comprising a carrier having a transmittance effective to transmit light of wavelengths absorbable by the photosensitizer.

17. The method of claim 16 wherein said carrier comprises (i) at least one of propylene glycol, polyethylene glycol, ethanol, glycerin, and (ii) a guar hydroxypropyl derivative.

18. The method of claim 16 wherein said carrier comprises at least one of propylene glycol, polyethylene glycol, ethanol, glycerin.

19. The method of claim 16 wherein said carrier comprises a guar hydroxypropyl derivative.

20. The method of claim 16 wherein the carrier comprises propylene glycol.

21. The method of claim 16 wherein the carrier comprises polyethylene glycol.

22. The method of claim 16 wherein the carrier comprises ethanol.

23. The method of claim 16 wherein the carrier comprises glycerin.

24. The method of claim 16 wherein the carrier is part of the composition to be used in step (a) and is selected from a group comprising a gel, cream, and paste.

25. The method of claim 24 wherein the carrier is a gel.

26. The method of claim 24 wherein the carrier is a cream.

27. The method of claim 24 wherein the carrier is a paste.

28. The method of claim 16 wherein the carrier is part of the composition to be used in step (b) and is selected from a group comprising a mouthwash, oral rinse, or atomizing spray.

29. The method of claim 28 wherein the carrier is a mouthwash.

30. The method of claim 28 wherein the carrier is an oral rinse.

31. The method of claim 28 wherein the carrier is an atomizing spray.

32. The method of claim 16 wherein the composition comprising the photosensitizer of step (b) is less viscous than the composition comprising the photosensitizer of step (a).

33. The method of claim 1 wherein the light used in step (d) has a peak wavelength ranging from about 610 nm to about 690 nm.

34. The method of claim 33 wherein the light used in step (d) has a peak wavelength ranging from about 620 nm to about 640 nm.

35. The method of claim 33 wherein the light used in step (d) has a peak wavelength ranging from about 650 nm to about 680 nm.

36. The method of claim 1 wherein the intensity of the light used in step (d) ranges from about 10 mW/cm² to about 200 mW/cm².

37. The method of claim 36 wherein the intensity of the light used in step (d) ranges from about 25 mW/cm² to about 100 mW/cm².

38. The method of claim 37 wherein the intensity of the light used in step (d) ranges from about 95 mW/cm² to about 105 mW/cm².

39. The method of claim 38 wherein the light dose used in step (d) ranges from about 2 J/cm² to about 60 J/cm².

40. The method of claim 39 wherein the light dose used in step (d) ranges from about 2 J/cm² to about 15 J/cm².

41. The method of claim 40 wherein the light dose used in step (d) ranges from about 6 J/cm² to about 12 J/cm².

42. The method of claim 1 wherein the microbial organisms are chosen from Porphyromonas gingivalis 381; Prevotella intermedia 25611; Actinobacillus actinomycetemcomitans UT32; Fusobacterium nucleatum 1213; and Bacteroides forsythus 43037 and combinations thereof.

43. A method of treating halitosis comprising the steps of:
(a) applying a photosensitizer to the interior of the mouth external to the periodontal pockets and including the tongue, buccal mucosa and gum regions;
(b) waiting a predetermined period of time; and
(c) irradiating the whole interior of the mouth with a non-coherent light having a wavelength spectrum absorbable by the photosensitizer, and at a predetermined light intensity and for a predetermined time period sufficient to deliver a predetermined light dose.

44. The method of claim 43 wherein steps (a) through (c) are repeated at predetermined intervals until the symptoms of halitosis are reduced to a desired level or eliminated.

45. A kit for treating microorganisms in the oral cavity comprising:
(a) an effective concentration of a photosensitizer;
(b) at least one light emitting treatment device operable to emit non-coherent light at a wavelength spectrum absorbable by the photosensitizer and at a predetermined light intensity;
(c) instructions for performing the method of claim 1, including instructions concerning the time of irradiation in step (d) of the method whereby the predetermined light dose is achieved in each of these steps.

46. A kit for treating halitosis in the oral cavity comprising:
(a) an effective concentration of a photosensitizer;
(b) at least one light emitting treatment device operable to emit non-coherent light at a wavelength spectrum absorbable by the photosensitizer and at a predetermined light intensity;
(c) instructions for performing the method of claim 43, including instructions concerning the time of irradiation in step (c) of the method whereby the predetermined light dose is achieved in each of these steps.

47. The kit of claim 45 or 46 comprising a member for administering the photosensitizer consisting of a syringe or cannula.

48. The use of a light emitting treatment device operable to emit non-coherent light in combination with a photosensitizer to inactivate microorganisms throughout the entire mouth, said light having a wavelength spectrum absorbable by the photosensitizer, and a peak wavelength ranging from about 610 nm to about 690 nm.

49. The use of claim 48, wherein the microorganisms cause halitosis.
